(19) United States
(12) Patent Application Publication
(10) Pub. No.: US 2010/0059049 A1
(43) Pub. Date: Mar. 11, 2010

(54) Dry-Powder Inhaler

(76) Inventor: Amir Genosar, Lafayette, CO (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 10/583,878

(22) PCT Filed: Jun. 5, 2006
(86) PCT No.: PCT/IL06/00647

§ 371 Date: Jan. 16, 2009

(30) Foreign Application Priority Data
May 16, 2006 (IL) 175664

(51) Int. Cl. A61M 15/00

(52) U.S. Cl. 128/203.15

(57) ABSTRACT

The invention provides a dry-powder inhaler device 10 comprising at least one air inlet 13, a flow chamber 11 and an air outlet 16 leading to a mouthpiece 12, the flow chamber 11 further comprising at least one compressed-powder volumes 19 and a multiplicity of scraping surfaces 18; wherein the inhalation action of the patient applied at the air outlet 16 causes air to flow from the at least one air inlet 13 through the flow chamber 11, the air flow generating relative motion between the compressed-powder volume 19 and the scraping surfaces 18 such that fine particles of powder are scraped from the compressed-powder volume 19 and inhaled by the patient.

### FIELD OF THE INVENTION

This invention relates to a medical device for dry-powder drug inhalation. Specifically, the present invention is a single-step inhaler where the act of inhalation releases the powder from a compressed solid form so that it can be inhaled into the lungs.

### BACKGROUND OF THE INVENTION

Numerous drugs, medications and other substances are inhaled into the lungs for rapid absorption in the blood stream. Inhaled drugs fall into two main categories: (1) liquids, including suspensions; and (2) powders. The present invention relates to the latter category.

Dry-powder inhalers need to deliver a particle size that is predominantly below 5 microns for maximum effectiveness. Such small particles are, however, thermodynamically unstable due to their high surface area to volume ratio, which provides significant excess surface free energy and encourages particles to agglomerate. In the inhaler, agglomeration of small particles and adherence of particles to the walls of the inhaler are problems that result in the active particles leaving the inhaler as large agglomerates or being unable to leave the inhaler and remaining adhered to the interior of the inhaler. In an attempt to improve that situation, dry powders for use in dry powder inhalers often include particles of an excipient material mixed with the fine particles of active material. Fine particles of active material suitable for pulmonary administration have often been prepared by milling, for example, jet milling. However, once the particles reach a minimum size referred to as the critical size, they re-combine at the same rate as being fractured, or do not fracture effectively and therefore do not reduce further in size. Thus, manufacture of fine particles by milling can require much effort and there are factors, which consequently place limits on the minimum size of particles of active material which can be achieved, in practice, by such milling processes.

Accordingly, one approach to the issue of maintaining a sub 5 micron particle from dry-powder type inhalers is to use particles of an excipient material mixed with the fine particles of the active ingredient. For example, published application 20040037785 describes a method of making particles for use in a pharmaceutical composition for pulmonary administration, the method comprising a milling step in which particles of active material are milled in the presence of particles of an additive material which is suitable for the promotion of the dispersal of the composite active particles upon actuation of an inhaler.

Another approach to this problem which is addressed in the prior art is the production of the requisite powder size by means of scraping from a compressed-powder directly before inhalation. U.S. Pat. No. 5,617,845 describes an inhalation device free from propellent gas with a storage chamber for a powdered substance to be inhaled. This device employs a trigger-operated pump which can be manually primed before the inhalation process by means of a button and which can be actuated in synchronism with the breathing, thereby generating a current of foreign air which disperses the metered substance. In this device, metering is carried out by means of a specially shaped metering notch as a metering chamber in the metering punch, which is rotated past a slightly compressed-powder charge. In this device, although compressed-powder is used, the step of scraping away the powder from the compressed-powder is a preparatory step, where the inhalation of the powder dose is then performed in a second and separate step. In further prior art, U.S. Pat. No. 5,887,586 describes a dry-powder aerosol generator, which is connected to a removable nose mask via a conduit system. The aerosol generator comprises a scraping mechanism, by means of which powder can be scraped off a tablet of compressed-powder, as well as means for aerosolizing the scraped-off powder in an air flow. Further prior art includes the Turbuhaler inhaler device (AstraZeneca PLC, London, UK) in which a dose of drug is scraped from a solid micronized drug matrix by the patient twisting the base of the device, prior to inhalation. However, in all these cases, the act of inhalation does not by itself cause the fine powder to be scraped away from the tablet of compressed-powder. On the contrary, the scraping of powder from a reservoir of compressed-powder is a separate process, and one in which complex generator elements are sometimes required.

This separation of the scraping process from the inhalation process in turn leads to two additional problems with prior art devices: (1) the powder tends to spill prior to use if the inhaler is shaken; and (2) the dose may be lost if the user blows into the device rather than inhales.

In view of these drawbacks and limitations of the prior art, what is needed is a simple and inexpensive inhaler without gas or other complex generators, capable of consistently delivering predominantly sub 5 micron particle sizes.

Therefore, it is an object of the invention to provide a simple, breath-powered inhaler where the act of inhalation causes the dry-powder to be scraped off a compressed-powder volume.

It is a further object of the invention to provide a convenient and portable housing for said inhaler.

It is a still further object of the invention to provide said specially designed device in a credit-card format.

It is a still further object of the invention to provide an ergonomic mouthpiece for miniature device, where said mouthpiece can be stored within a credit-card format device.

It is a still further object of the invention to provide a dry-powder inhaler which synchronizes the drug release with the inhalation action of the patient, while spreading the delivery over a defined duration of the breath and controlling for particle size.

It is further the object of the invention to provide a device that enables the transporting of the drug separate from the device such that the patient can load said drug into the device.

It is further the object of the invention to provide a device that is indifferent to accidental air-blow into the device.

These and other objects of the present invention are achieved in the preferred embodiments disclosed below by providing a breath-powered dry-powder inhaler.

### SUMMARY OF THE INVENTION

The inhaler device of the present invention provides an improved and simplified mechanism for dry-powder drug inhalation, which ensures the synchronization of fine-particle release during inhalation. The operating principle of said device is that the act of inhalation itself causes fine powder to be scratched or rubbed away from the surface of a compressed-powder volume, where the thus released powder is inhaled directly. Advantageously, such an approach is inherently free of the problems of prior art devices where a powder dose can be spilled or where exhaling into the device can disturb the powder. As the powder for inhalation is only produced during the inhalation, the synchronization of the powder inhalation with the breath is achieved inherently in this design. Depending on the drug type, said synchronization with the inhalation curve is extremely important in order to ensure that the drug is delivered to the required areas of the lungs. Thus, for several drugs, a too early or too late delivery results in extremely low efficiency of the administration, which in turn can affect the results of the treatment and even limit the use of certain devices from critical drugs. Additionally, in many cases, a pre-determined delay of the drug discharge to a certain point in the inhalation curve and the release of the drug over a defined period of that inhalation curve (rather than in a bolus) provides optimal results. By having the drug in a one-piece form in the device the management of the drug in the device become simpler and thus enables a simpler and more compact mechanism.

The compressed-powder of the present invention shall refer to any form of drug, vaccine or other therapeutic agent in which a powder is formed into a solid matrix. Said powder may be any kind of powder cake such as a freeze dried cake, or any kind of powder or micronized powder bonded or otherwise arranged into a solid matrix.

The inhaler device of the present invention is a dry-powder inhaler device comprising at least one air inlet, a flow chamber and an air outlet leading to a mouthpiece, said flow chamber further comprising at least one compressed-powder volume and at least one scraping surface; wherein the inhalation action of the patient applied at said air outlet causes air to flow from said at least one air inlet through said flow chamber, said air flow generating relative motion between said at least one compressed-powder volume and said at least one scraping surface such that fine particles of powder are scraped from the compressed-powder volume(s) and inhaled by the patient.

In preferred embodiments of the present invention, said dry-powder inhaler device comprises a multiplicity of air inlets.

In further preferred embodiments of the present invention, said dry-powder inhaler device comprises a multiplicity of compressed-powder volumes.

Preferably the scraping surface is a blade of an impeller, said blade gradually extending outwards as said impeller rotates, thereby ensuring a time lag between the start of said inhalation action and the first release of said fine particles.

Said device preferably further comprises (a) a particle filter located between said flow chamber and said outlet to ensure that large particles are not inhaled and (b) a mouthpiece attachable to said outlet. Said mouthpiece may either be an integral part of said inhaler device or may be attached by the patient to said outlet. In the latter case, the inhaler device may further comprise a storage compartment for said mouthpiece. Regarding the scraping surfaces and the compressed-powder volumes, either one is static and the other movable, or both or movable.

The inhaler device is preferably shaped like a credit-card, a conventional hand-held inhaler, or have any other ergonomically suitable shape, including that of a cylinder, a prism, a disk, and an oval.

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### BRIEF DESCRIPTION OF THE DRAWINGS

FIG. 1 presents isometric and planar views of a single use disposable credit-card shape embodiment of the invention where the compressed-powder is static;

FIG. 2 presents isometric views of a multiple-use credit-card shape embodiment of the invention where the compressed-powder is static;

FIG. 3 presents isometric and cross-sectional views of an embodiment of the invention where the compressed-powder is the moving element; and

FIG. 4 presents isometric, planar and cross-sectional views of an embodiment of the invention where the compressed-powder is embedded on the film walls of the flow control chamber.

### DETAILED DESCRIPTION OF THE DRAWINGS

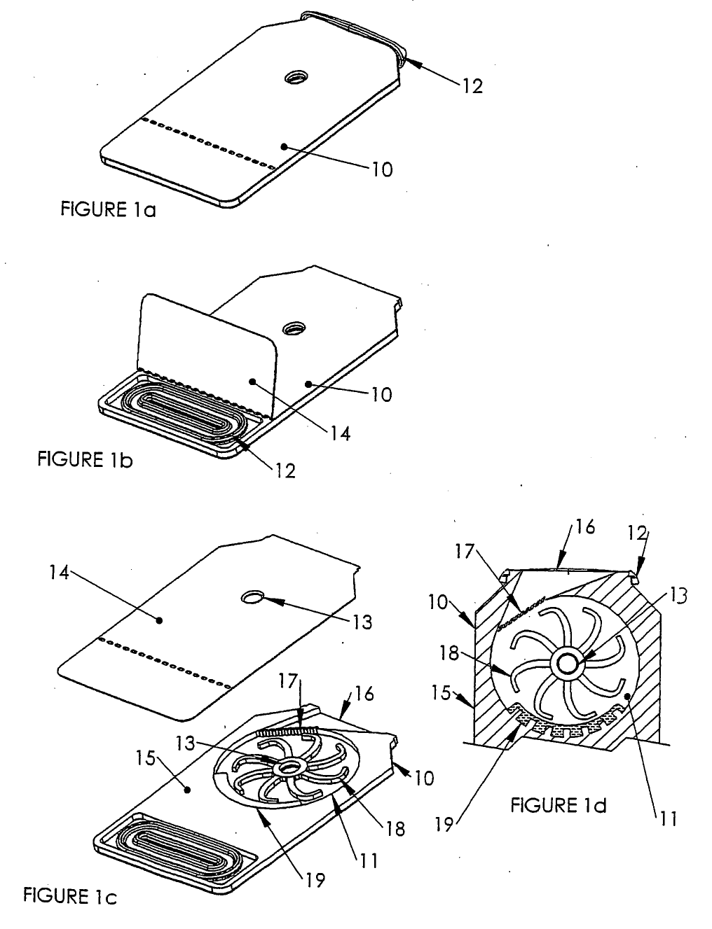

Referring now to FIG. 1, a preferred embodiment of the device of the present invention is shown, in which a credit-card style design is employed. FIG. 1a provides an overall isometric view of this preferred embodiment, shown ready for use, with a rubber mouthpiece 12 shown attached around the outlet 16. As an inhaler 10 of this size and shape may not be convenient to place in the mouth, said rubber mouthpiece 12 is provided along with the inhaler device 10, and is preferably stored in a dedicated compartment thereof, as shown in FIG. 1b. To use the mouthpiece 12, it is extracted from said compartment and stretched around the drug outlet 16. By so doing, the mouthpiece 12 deforms into the ergonomically advantageous shape shown in FIG. 1a. The advantage of this approach is that the device can easily be carried in a credit-card slot in a wallet, while keeping the mouthpiece from getting contaminated. It is also easier to clean and wash such a removable mouthpiece 12. Said mouthpiece 12 can be made from elastic polymers such as Silicone Rubber or Santoprene®. Alternatively, the outlet 16 can be tapered into a narrow form (not shown) such that it can be inserted into a mouth more comfortably. In such a case, the need for a separate mouthpiece 12 can be obviated.

Referring now to FIG. 1c, an exploded isometric view of a preferred embodiment of the inhaler device 10 of the present invention is provided. Said device 10 comprises a body 15 sandwiched between two film walls 14, where at least one of said walls 14 further comprises an air inlet 13. Said film walls 14 may comprise multi-layer plastic film and/or metalized plastic films. Advantageously, this construction enables the device 10 to benefit from the strength and excellent barrier properties that such film walls possess. The body 15 of said inhaler device 10 further comprises a flow chamber 11 containing an inhalable drug in a compressed-powder 19 form, an impeller 18, and an outlet filter 17. When air is inhaled by the patient, the resulting air flow from the air inlet 13, via the flow chamber 11 and out through the outlet filter 17 to the outlet 16, causes the impeller 18 to rotate. By appropriate relative arrangement of the impeller 18 and the compressed-powder and the employment of flexible blades on the impeller 18, said rotation causes said blades to stretch out such that the tip of one or more impeller 18 blades come in contact with the compressed-powder 19, causing said tip or tips to scrape powder off said compressed-powder 19. In this embodiment, said volume of compressed-powder 19 is adhered to the inner circumferential wall of the flow chamber 11. By controlling the flow parameters, and the impeller mechanical parameters a control on the delay and duration of the drug release can be achieved, resulting in synchronization with the inhalation cycle. Additionally, by controlling the mechanical properties of the impeller 18 and in particular the mass and flexibility of the blades and the roughness of their tips and the properties of the compressed-powder 19, the characteristics of the powder generated and inhaled can be controlled. The outlet filter 17 prevents large size powder particles from reaching the patient. Advantageously, the overall result of this mechanism is the provision of a breath-powered, controllably-delayed drug delivery which can be sustained during the breath of the patient.

In the above preferred embodiment, the impeller 18 can be made from injection-molded thermoplastic materials such as polyurethane or polycarbonate, or alternatively from sheet metal spring materials. The outlet filter 17 can be either be an integrally-formed part of the body 15, or a separate component such as a Porex™ piece (from Porex Corporation, Fairburn, Ga., USA) or a non-woven mesh. A centrifugal separation technology can be combined to allocate the large particles to specific area. Additionally, it will be obvious to one skilled in the art that the outlet filter 17 can be designed in many shapes and structures. For example, said outlet filter 17 can extend further than shown around the circumference of the flow chamber 11, where the air passing through said filter 17 is channeled to the outlet 16. Due to the use of film walls 14 with good barrier properties, provided that a seal (not shown) to the inlet 13 and outlet 16 is maintained in between uses, the compressed-powder 19 will be maintained in an environment which protects it against humidity and other pollutants. It will also be obvious to one skilled in the art that a mechanical restrictor can be implemented in the flow chamber 11 such that it will prevent the impeller 18 from turning backwards, thereby preventing accidental wasting of the drug.

Referring now to FIG. 1d, a planar view of a slight modification of this embodiment is provided. Whereas in FIG. 1c the compressed-powder 19 is adhered to the inner circumferential wall of the flow chamber 11, in this preferred embodiment said compressed-powder volume 19 is attached via teeth to the plastic forming the body 15 at that same location. The compressed dry powder 19 may comprise any inhalable drug/carrier combination known in the field of drug tablets manufacturing, whether cold compressed into a solid form or otherwise. It is obvious to those skilled in the art that the dispersion of drug powder in the matrix powder is controllable. For example, the powder (in the “teeth”) that will remain unused in the present embodiment would preferably not contain any of the active drug ingredients. The compressed drug can be implemented on the chamber walls as one piece by mechanical atachement or impregnated on the walls or on a separate part that is introduced to the chamber such as a film strip.

Whereas in the above embodiment, the blades of the impeller 18 extend out toward the compressed-powder during use, in an alternative embodiment, the blades of the impeller 18 could remain fixed while the compressed-powder 19 is spring-loaded to press forward into said blades or otherwise forced advanced toward the impeller. For example a special mechanism can advance the dug toward the impeller in response to the pressure in the chamber or the speed of the impeller or the rotations of the impeller.

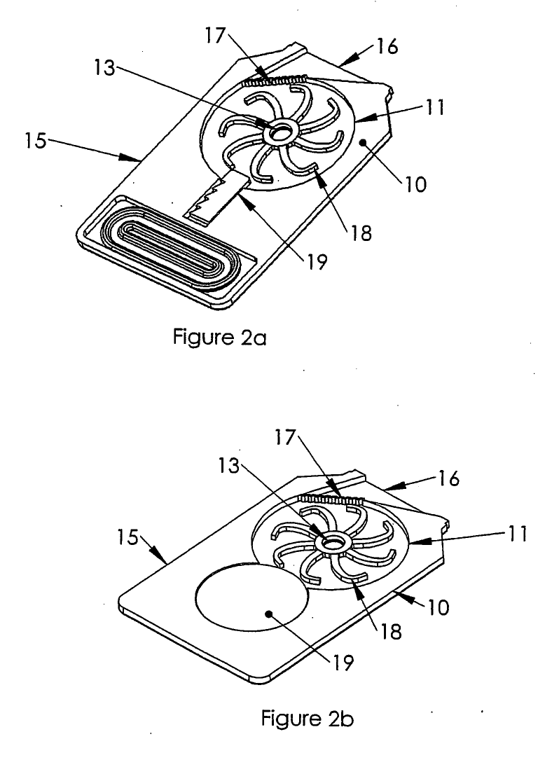

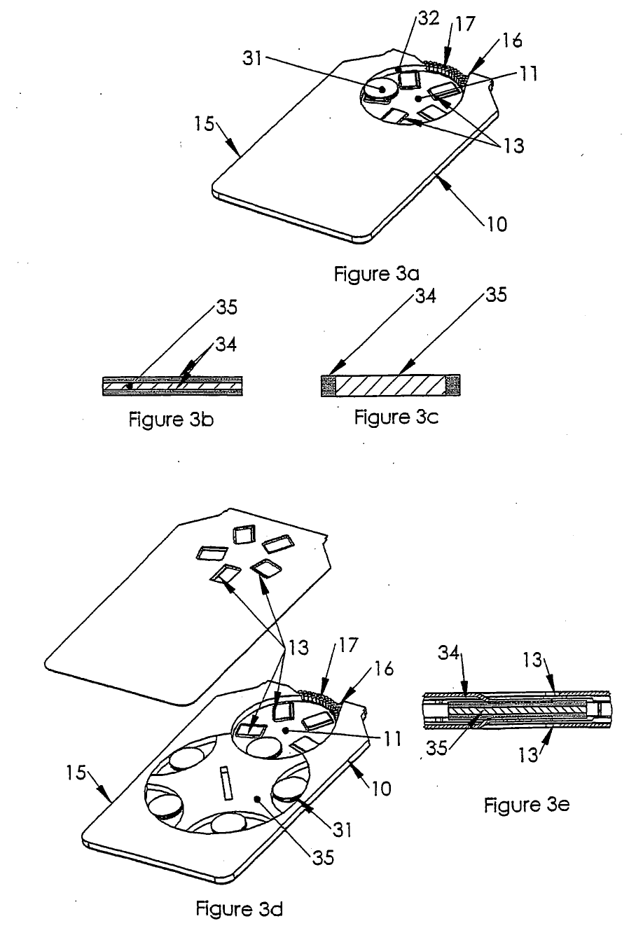

The inhaler device 10 of the present invention may be provided in either disposable or multiple-use embodiments. Referring now to FIG. 2, a multiple-use variant of the approach described in connection with FIG. 1 above is presented. FIG. 2a shows an isometric view of an embodiment in which the compressed-powder 19 is in a shape of a bar that can be incrementally advanced into the flow chamber 11 by a special mechanism (not shown) that engages with the ratchet teeth. Similarly, FIG. 2b provides an isometric view of an embodiment in which the compressed-powder 19 is in a shape of a disk that can be rotated between uses, in order to expose another section of said disk to the impeller 18 each time. In this case, the compressed-powder 19 disk can be completely made of compressed-powder or alternatively it can have a carousel structure comprising a rigid framework with compressed-powder volumes located at several points on its circumference. Said rigid structure is preferably formed from plastics such as polypropylene. Due to the inexpensive nature of the design employed in FIGS. 1 and 2 above, the device 10 presented can be a disposable one, whether intended for multiple-use or single use. Alternatively, the device 10 can be designed so that the compressed-powder 19 can be replaced by the user, thus making the device a permanent multiple-use device. Advantageously, by enabling the patient to replace the compressed-powder 19, the device 10 can be delivered separately from the drug to the user, pharmacist, or physician; thereby widening the flexibility of the drug distribution model. In a further multiple-use embodiment (not shown), the compressed-powder volumes are stored individually in a strip, said strip being advanced toward the scraping surface(s) and the powder exposed, as each next dose is required.

Whereas FIGS. 1 and 2 present an embodiment wherein the compressed-powder drug 19 is static and is ground into a powder by an element moving against it; referring now to FIG. 3 a further preferred embodiment is illustrated in which the compressed-powder is the moving element and the powder is scraped away as said compressed-powder moves against the static circumferential walls 32 of the flow chamber 11. This embodiment employs a plurality of air inlets 13 in the form of cantilevered sections of the film walls 14 that enclose the body 15, arranged such that said air inlets 13 impart a swirling air flow motion to the flow chamber 11 and thereby make the disk 31 spin around said chamber 11. The exploded isometric diagram of FIG. 3d shows a preferred arrangement for such slots in the upper film wall 14, said arrangement being mirrored in the lower film wall as shown in the figures. Referring now to FIG. 3a, an isometric view of a single-use embodiment of this approach is shown, in which the air entering from the inlet 13 flows rotationally around the flow chamber 11 and then through the outlet filter 17 to the outlet 16. Said rotational air flow causes a compressed-powder disk 31 to rotate along the circumferential wall 32 of the flow-chamber 11, thereby generating fine powder due to the friction between said wall 32 and said disk 31. Such disks 31 can be replaced through the air inlet 13. In this figure the disk 31 is entirely fabricated from compressed-powder, and as described above, the active ingredients can be controllably concentrated in the outer layer of said compressed-powder. Other possibilities for the fabrication of said disk 31 include (a) the employment of a hard core 35 in the shape of a thin disk covered by upper and lower layers of compressed-powder 34 (as shown in FIG. 3b), and (b) a hard core 35 in the shape of a disk whose circumference is covered by a layer of compressed-powder 34 (as per FIG. 3c). The former embodiment produces fine powder as the disk 31 scratches against the flat walls of the flow chamber 11, said walls being made suitable rough. Similarly, the latter embodiment produces fine powder as the disk 31 scratches against the circumferential wall 32 of the flow chamber 11, said wall being made suitably rough. Referring now to FIG. 3d, a further preferred embodiment of the inhaler device of the present invention is shown, in which a carousel component 36 serves to contain a multiplicity of the above described disks 31, such that a new disk 31 can be exposed to the flow chamber 11 at each turn of the carousel 36. In this embodiment, exhausted disks can either be manipulated back to the carousel 36 or can be disposed of via one of the air inlet holes 13. Referring now to FIG. 3a, a cross-sectional view is provided of the inhaler device of the present invention, in which a compressed powder disk as per FIG. 3b is shown in contact with the above-described cantilevered sections of the film walls of the device which are serving to form the air inlets 13. In this embodiment, the inner side of said sections serves to scrape off powder 34 from the disk by frictional action against said disk.

While FIGS. 1-3 presented powder generation of a static element working against a moving element, one of them being the compressed-powder, it will be obvious to those skilled in the art that the powder can also be generated by the interaction of two moving elements and such an embodiment is included in the present invention providing that such interaction is driven by the inhalation.

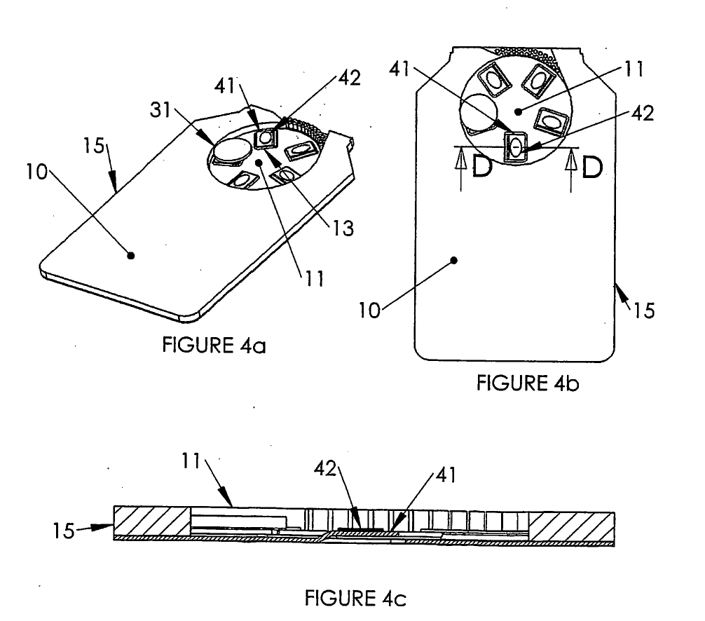

Referring now to FIG. 4a an isometric view of a further preferred embodiment of the inhaler device of the present invention is shown. In this embodiment the compressed-powder 42 is embedded in or otherwise deposited on lowered sections 41 of the flat film wall 14 covering the flow chamber 11. As per FIG. 3 above, said film wall 14 is preformed and cut in a way that these lowered sections 42 of the wall 14 have the shape of flexible fingers, whose embedded powder 42 areas lightly touch the rotating disk. As per the previous embodiment, said sections also serve to swirl the incoming air so that the disk 31 rotates around the flow chamber 11. The difference from the previous embodiment is that in this case the disk does not contain a compressed-powder but only serves to scrape off the drug powder from said embedded powder areas 42. Referring now to FIG. 4b, a planar view of this preferred embodiment is presented in order to show the line D-D represented by the cross-sectional view shown in FIG. 4c. Referring now to FIG. 4c, a disk 31 is shown on the left hand side, and the impregnated compressed-powder area 42 that it will scratch against on contact is shown on the inside of the cantilever structure 41.

While the above embodiments describe credit-card shape designs, it will be obvious to one skilled in the art that a number of device designs are possible, including a range of solutions for compressed-powder arrangements, and loading and replacing solutions. For example, the device may be in the shape of a prism, a disk, an oval, or use the form-factor of existing, conventional hand-held inhalers; providing only that the internal volume is sufficient to allow the breath-powered scraping or rubbing action to liberate the fine powder as described above. It should also be apparent that the device of the present invention can further incorporate a number of standard drug-dosing device components or functions known in the art. These elements include a child-proof mechanism to protect against inadvertent activation by a child; a counter display showing the number of inhalations, shipping seals, air-tight resealing plugs, etc. Further, it will be obvious to one skilled in the art that a number of drugs can be inhaled simultaneously using the device of the present invention, whether by employing a multiplicity of compressed-powder drug volumes where different volumes contain different drugs, or by means of mixing a multiplicity of drugs within any given compressed-drug volume. Additionally, where a “magazine” of compressed drug volumes is used as per FIG. 2a or FIG. 3d, each of said volumes may comprise a different drug or different drug combination. Advantageously, said arrangement enables the sequential administration of a number of drugs.

It will also be obvious to those skilled in the art that, although primarily a breath-powered device, it is possible to use a source of auxiliary power to assist in the scraping action and thereby increase its efficiency. Said auxiliary power means include the use of a lever with a spring that will add auxiliary force to the impeller, a compressed gas cylinder and an electric motor. Said auxiliary power source may be incorporated with in the device, or alternatively this power source can be external. For example by implementing a static magnet in the impeller of FIG. 1 the impeller could gain power from an external electric or magnetic field. In another configuration the impeller can incorporate a non-magnetic electric conductive material that will be driven from an alternating magnetic field by means of an Eddie Current drive arrangement.

A dry-powder inhaler is described above. Various details of the invention may be changed without departing from its scope. Furthermore, the foregoing description of the preferred embodiment of the invention and the best mode of practicing the invention are provided for the purpose of illustration only and not for the purpose of limitation—the invention being defined by the claims.

1. A dry-powder inhaler device comprising at least one air inlet, a flow chamber and an air outlet leading to a mouthpiece, said flow chamber further comprising at least one compressed powder volume and at least one scraping surface; wherein the inhalation action of the patient applied at said air outlet causes air to flow from said at least one air inlet through said flow chamber, said air flow generating relative motion between said at least one compressed-powder volume and said at least one scraping surface such that fine particles of powder are scraped from the compressed-powder volume and inhaled by the patient.

2. The inhaler device of claim 1 where said scraping surface is a blade of an impeller, said blade gradually extending outwards as said impeller rotates, thereby ensuring a time lag between the start of said inhalation action and the first release of said fine particles.

3. The inhaler device of claim 1 where said compressed-powder volume is divided into a number of sections that can be advanced into said flow chamber in order to reload said device.

4. The inhaler device of claim 1 further comprising a particle filter located between said flow chamber and said outlet to ensure that large particles are not inhaled.

5. The inhaler device of claim 1 further comprising a mouthpiece attachable to said outlet.

6. The inhaler device of claim 5 where said mouthpiece is an integral part of said inhaler device.

7. The inhaler device of claim 5 where said mouthpiece is attached by the patient to said outlet.

8. The inhaler device of claim 7 further comprising a storage compartment for said mouthpiece.

9. The inhaler device of claim 1 where said scraping surfaces are movable and said compressed-powder volumes are static.

10. The inhaler device of claim 1 where said scraping surfaces are static and said compressed-powder volumes are movable.

11. The inhaler device of claim 1 where both scraping surfaces and said compressed-powder volumes are movable.

12. The inhaler device of claim 9 where the movement is synchronized with the inhalation flow.

13. The inhaler device of claim 1 where said device is shaped like a credit-card.

14. The inhaler device of claim 1 where the shape of said device belongs to the group including cylinders, prisms, disks, ovals, and conventional hand-held inhalers.

15. The inhaler device of claim 1 where said compressed-powder volume belongs to the group including disks, tablets, and fixed internal surfaces of said device.

16. The inhaler device of claim 15 where the drug is fixed to a member introduced to the flow control chamber such as a film strip.

17. The inhaler device of claim 16 where the drug is protected in enclosures that are opened prior to use.

18. The inhaler device of claim 1 where the active drug ingredients are selectively dispersed in the compressed-powder volume.

19. The inhaler device of claim 1 where said compressed-powder volume is compressed into a structural element.

20. The inhaler device of claim 1 where said device is loaded with more than one drug.

21. The inhaler device of claim 1 where said compressed-powder volume is replaceable.

22. The inhalation device of claim 1 where said scraping means belongs to the group including impellers and fixed internal surfaces of said device.

23. A dry powder inhaler device according to claim 1 comprising a multiplicity of air inlets.

24. A dry powder inhaler device according to claim 1 comprising a multiplicity of compressed-powder volumes.

25. A dry powder inhaler device according to claim 1 comprising a multiplicity of scraping surfaces.
